(19) United States
(12) Patent Application Publication
(10) Pub. No.: US 2005/0010187 A1
(43) Pub. Date: Jan. 13, 2005

(54) Inter-labium pudenda pad and package body for the pad

(75) Inventors: Satoshi Mizutani, Kagawa (JP); Koichi Yamaki, Kagawa (JP); Yuki Noda, Kagawa (JP)

Correspondence Address: DARBY & DARBY P.C., P. O. BOX 5257, NEW YORK, NY, 10150-5257, US

(73) Assignee: Uni-Charm Corporation, Kawanoe-Shi (JP)

(21) Appl. No.: 10/888,540

(22) Filed: Jul. 8, 2004

Related U.S. Application Data
(63) Continuation of application No. PCT/JP02/12017, filed on Nov. 18, 2002.

(51) Int. Cl. A61F 13/15; A61F 13/20

(52) U.S. Cl. 604/385.101; 604/385.17

(57) ABSTRACT

The present invention relates to an interlabial pad used by engaging with labia of a woman, especially, an interlabial pad to be used with a sanitary napkin, and a wrapping body where the interlabial pad is enclosed in a wrapping container for individual wrapping, and provides an interlabial pad that can prevent the side leakage and preserve the cleanliness function inherent in the interlabial pad and, furthermore, improve its absorption conservation capacity by far.

An interlabial pad, comprising an absorbent sheet body where a water permeable face side sheet 11 and a water permeable or impermeable back face side sheet 12 are bonded in such a manner enclosing an absorbent body 13 for absorbing body fluid, provided with a body-fluid receiving hole 14b formed in the vicinity of the middle portion, the body-fluid receiving hole having a recess with a bottom portion corresponding to the garment side, and the size of the opening thereof being such a size that can fit to a pair of labia minora in a way to envelop them from outside.

### CROSS-REFERENCE TO RELATED APPLICATIONS

This application is a Continuation of International Application No. PCT/JP2002/012017 filed Nov. 18, 2002, which application published in Japanese on Jun. 3, 2004 as WO 2004/045478 A1 under PCT Article 21 (2).

### FIELD OF THE INVENTION

The present invention relates to an interlabial pad to be used by engaging in the female labia, especially, an interlabial pad that can be used with a sanitary napkin, as well as, a wrapping body wherein the interlabial pad is enclosed in a wrapping container for individual wrapping.

### BACKGROUND ART

Conventionally, as a sanitary article for women, sanitary napkins (referred to simply as “napkin”, hereinafter) and tampons are generally used. However, napkins are applied to the garment for use, and often let the menstrual blood leak from the gap in respect to the body, as they are hardly adhered to the vicinity of the ostium vaginae, while tampons generate often a foreign feeling or a discomfort during the wearing, due to the attribute of that article, and it was difficult to fix into the vagina.

Under such situation, in recent years, a sanitary article called an interlabial pad has attracted attention, as a sanitary article positioned between the napkin and the tampon.

This interlabial pad is the one to be fitted by pinching a part thereof between interlabia of a woman, and applying to the labia, characterized by that the menstrual blood leaks hardly because it is closely adhered to the body, and the psychological resistance on wearing is low compared to the tampon to be inserted into the vagina.

As for the interlabial pad mentioned above, for instance, an interlabial pad 24 as shown in FIG. 2 has been proposed (the Japanese Patent Laid-Open No. 1986-149145).

Such interlabial pad 24 is used by positioning a forward area 24a of the pad on the body front side, positioning a backward area 24b of the pad on the body back side, thereafter, forming a convex portion by bending the backward area 24b of the pad substantially along an central line 26 in the longitudinal direction of the pad, pinching the convex portion between interlabia, and fixing by the pinching force of the labia themselves. Such interlabial pad 24 can afford a comfortable feeling of use, without making the wearer be aware of its fitting, because the contact area with the body (or friction with the body) out of the labia is extremely small compared to the napkin. In addition, the interlabial pad 24 is also excellent in cleanliness function, because it absorbs directly menstrual blood mainly inside the labia, and menstrual blood does not attach in a wide range from the vestibule portion to the haunches as in the case for the napkin.

However, as for the aforementioned interlabial pad 24, an upper limit is imposed on the volume of the convex portion to be pinched between interlabia, for the reason of prevention of giving a foreign feeling to the wearer. Consequently, the quantity of menstrual blood absorbed and held is necessarily less than that of the napkin, and menstrual blood filtrates easily outside the interlabial pad 24.

Besides as the aforementioned interlabial pad 24 is smaller in size than the napkin, menstrual blood often leaks from the side. As sanitary article effective for preventing the side leakage, a napkin 34, for example as shown in FIG. 3, has been proposed (the Japanese Utility Model Application Laid-Open No. 1995-13319).

Such napkin 34, is the one wherein an elastic and water impermeable flap 36 is attached to the body side of the napkin itself, and an adhesion portion 37 is formed on the body side surface of the flap 36. When the adhesion portion 37 is stuck to the body, a three-dimensional water impermeable barrier is formed between the body and the napkin itself, and the side leakage can be prevented effectively.

However, the aforementioned napkin 34 presents inconveniences similar to the napkin of the prior art. Namely, the inconvenience that the wearer can not enjoy a comfortable feeling of use is not resolved at all, because menstrual blood attaches in a wide range from the vestibule portion to the haunches and, furthermore, the size is larger than the interlabial pad and bulky.

The present invention was devised in view of the aforementioned inconveniences and has an object of providing an interlabial pad that can prevent the side leakage, preserve the cleanliness function inherent to the interlabial pad, and further, enhance its absorption conservation capacity remarkably. In addition, the present invention consists in providing a wrapping body that can enclose such interlabial pad compactly, as well as, take it out so that its function can be deployed immediately.

### DISCLOSURE OF THE INVENTION

In order to solve the aforementioned inconveniences, the interlabial pad of the present invention is characterized by that a body-fluid receiving hole is provided in the vicinity of the middle portion thereof. The absorption conservation capacity of the interlabial pad can be improved remarkably by affording the absorption ability to the body-fluid receiving hole by composing the same with an absorbent sheet body in this way. In addition, the attachment of body fluid to the body is prevented as much as possible, by leading menstrual blood or other body fluids to the body-fluid receiving hole, and thereby, the cleanliness function inherent to the interlabial pad can be enhanced.

(1) An interlabial pad, used by engaging with the labia, comprising: a water permeable face side sheet facing to a body side; a water permeable or impermeable back face side sheet facing to a garment side; and an absorbent body for absorbing body fluid wherein the face side sheet and the back face side sheet are bonded together to enclose the absorbent body and wherein a body-fluid receiving hole is provided in the vicinity of a middle portion of the interlabial pad, the body-fluid receiving hole being a recess having a bottom portion on the garment side, and having an opening with such a size that can fit a pair of labia minora so as to envelop them from outside.

The aforementioned interlabial pad is worn in a way to engage a pair of labia minora with an opening portion of the body-fluid receiving hole provided in the vicinity of the middle portion. When the interlabial pad is worn in this way, body fluid (especially menstrual blood) discharged from the ostinum vaginae is led rapidly to the body-fluid receiving hole and once stored. Then, the stored menstrual blood is absorbed by the absorbent body in the absorbent sheet body constituting the body-fluid receiving hole. The aforementioned interlabial pad, being provided with a recess portion that is the body-fluid receiving hole, can increase the volume and the surface area of the absorbent body as the whole pad, without increasing the volume of the portion to be pinched between interlabia. Consequently, the absorption conservation capacity of the interlabial pad can be improved remarkably.

Besides, by adopting such composition, menstrual blood is remained and absorbed in the body-fluid receiving hole entirely, so the labia and menstrual blood do not come into contact with for a long time as in the case for the napkin of the prior art, and menstrual blood does not attach in a wide range from the vestibule to the haunches, because menstrual blood is stored and absorbed exclusively in the interior of the body-fluid receiving hole. Consequently, it is possible to improve the cleanliness function inherent to the interlabial pad. What is more, menstrual blood that could not be absorbed will not flow along the surface of the interlabial pad, and leak from the side.

(2) The interlabial pad according to (1), wherein said body-fluid receiving hole projects toward the garment side of said interlabial pad.

As the aforementioned interlabial pad is the one where a body-fluid receiving hole 44b projects on the garment side of an interlabial pad 44, for example as shown in FIG. 4 and FIG. 5, it is possible to secure a capacity that can store a certain quantity of menstrual blood and, at the same time, menstrual blood and labia come hardly into contact, because a clearance is created between body fluid (especially menstrual blood) stored in the body-fluid receiving hole 44b and absorbed there and the labia. Consequently, the skin is seldom soiled, skin roughness can be reduced, and it becomes possible to further improve the cleanliness function inherent to the interlabial pad.

(3) The interlabial pad according to (1) or (2), wherein said body-fluid receiving hole is shaped so that the middle portion of said interlabial pad caves in.

As an example of the “hole projects toward the garment side of the interlabial pad”, the “one where the middle portion of the pad is shaped to cave in” for instance as shown in FIG. 6 can be cited. In such form, as the body-fluid receiving hole 44b comprises a cave-in portion of the absorbent sheet body 46, and a substantial flat area 44a and the body-fluid receiving hole 44b are formed integrally with a single absorbent sheet body 46, body fluid (especially menstrual blood) discharged in the vicinity of the opening portion of the body-fluid receiving hole 44 is also conducted smoothly to the body-fluid receiving hole 44b along a face side sheet 41 of the integrally formed absorbent sheet body 46. In this process, as menstrual blood is absorbed by an absorbent body 43 of the absorbent sheet body 46, a situation where menstrual blood leaks outside the pad can be avoided.

It should be noted that, the pad of such mode can be formed relatively easily by a method of sandwiching and compressing an absorbent sheet body 46 made by laminating a face side sheet 41, an absorbent body 43 and a back face side sheet 42 between a heated convex die 50 and a recess die 51 for instance as shown in FIG. 7.

(4) The interlabial pad according to (1) or (2), comprising: a sheet-shaped first absorbent sheet body having a through-hole of such a size that can fit to a pair of labia minora so as to envelop them from outside, and a bag-shaped second absorbent sheet body serving as a portion of said body-fluid receiving hole, wherein a garment side surface of said first absorbent sheet body and an inner side surface of the bag of said second absorbent sheet body are stuck in contact each other and stuck together.

Other than the one formed integrally by the aforementioned single absorbent sheet body, the “hole projects toward the garment side of the interlabial pad” may also be those formed from a plurality of absorbent sheets. “One comprising a sheet-shaped first absorbent sheet body 46a having a through-hole in the vicinity of the middle portion and a bag-shaped second absorbent sheet body 46b constituting the portion of body-fluid receiving hole 44b, wherein the garment side surface of the first absorbent sheet body 46a and the bag body inside surface of the second absorbent sheet body 46b are stuck in a state of contact”, for instance as shown in FIG. 8 can be cited. Such mode has an advantage that it can be manufactured extremely simply by a manufacturing method described below.

(5) A method of producing an interlabial pad of (4), comprising the steps of: folding the sheet-shaped first absorbent sheet body having, in the vicinity of the middle portion, the through-hole of such a size that can fit to a pair of labia minora so as to envelop them from outside substantially along a central line in the longitudinal direction; pinching said first absorbent sheet body by the sheet-shaped second absorbent sheet body folded substantially along the central line in the longitudinal direction in a way to seal the through-hole portion of said folded first absorbent sheet body; and forming a bag portion serving as the body-fluid receiving hole by sticking an abutment face of an outer peripheral side of the through-hole of in a surface of said first absorbent sheet body to an abutment face in a surface of said second absorbent sheet body.

The interlabial pad according to the aforementioned (4) can be manufactured extremely simply by a method shown below. First, a first absorbent sheet body having in the vicinity of the middle portion a through-hole of such size that fit a pair of labia minora in a way to envelop from outside is folded substantially along the central line in the longitudinal direction. This through-hole constitutes the opening portion of the body-fluid receiving hole afterward.

Then, as shown in FIG. 9, the first absorbent sheet body 46a is pinched, by a sheet-shaped second absorbent sheet body 46b, similarly folded substantially along the central line in the longitudinal direction, in such a way to seal the through-hole portion 46c of the folded first absorbent sheet body 46a.

Further, as shown in FIG. 10, the outer peripheral side of the through-hole 46c on the surface of the first absorbent sheet body 46a, a contact face 46d with the second absorbent sheet body 46b, and a mutual contact face 46c of surfaces of the second absorbent sheet body 46b are stuck with an adhesive or the like. At last, an interlabial pad 44 having a bag portion constituting the body-fluid receiving hole 44b, as shown in FIG. 8, can be obtained by opening the first absorbent sheet body 46a in the arrow direction as shown in FIG. 11. The interlabial pad 44 can be provided as product in a state cut into a desired shape.

(6) The interlabial pad according to (1) or (2), wherein said body-fluid receiving hole has a tucking portion where said absorbent sheet body is tucked in.

The aforementioned interlabial pad is easy to carry because it can be folded before using the pad, reducing the storage capacity and, in addition, it has an advantage of easy handling, because the body-fluid receiving hole is formed extremely easily by unfolding the folded pad for the use thereof.

(7) The interlabial pad according to (6), wherein said tucking portion is formed on a side wall of said body-fluid receiving hole.

The tucking portion may be formed in any portion of the absorbent sheet body constituting the body-fluid receiving hole and, for example as shown in FIG. 12, it can be formed on the side wall of the body-fluid receiving hole 44b. As the interlabial pad 44 of such shape can reduce the height dimension, by making the tucking portion 47 into the folded state, it is possible to store in a wrapping container in a compact state, even in case of making the body-fluid receiving hole large during the use of the pad. Consequently, it serves to the improvement of the body fluid (especially menstrual blood) absorption capacity (absorption quantity, absorption speed) by the pad, and the improvement of convenience of carrying and handling.

(8) The interlabial pad according to (6) or (7), wherein said tucking portion is formed on a bottom portion of said body-fluid receiving hole.

Other than on the side wall of the body-fluid receiving hole, for example as shown in FIG. 13 and FIG. 14, the tucking portion may also be formed on the bottom portion of the body-fluid receiving hole 44b. In short, the tucking portion can be formed both on the side wall and bottom portion of the body-fluid receiving hole, or only on the bottom portion. For instance, as for the interlabial pad 44 shown in FIG. 13 and FIG. 14, the tucking portion 47 is formed on the bottom portion of the body-fluid receiving hole 44b and the bottom portion of the body-fluid receiving hole 44b is in a situation to protrude toward the opening portion direction of the body-fluid receiving hole 44b.

Similarly to the interlabial pad according to the aforementioned (7), this structure also serves to the improvement of the body fluid (especially menstrual blood) absorption capacity (absorption quantity, absorption speed) of the pad, and the improvement of convenience of carrying and handling. In short, the formation of the tucking portion 47 allowing to improve the surface area of the body-fluid receiving hole 44b during the use, the menstrual blood absorption capacity of the interlabial pad 44 can be enhanced, and as show in FIG. 15, as the interlabial pad 44 can be stored by folding compactly before the use, it presents a good storage function, and becomes the one which is convenient for the wearer to carry. Besides, the handling is also extremely simple, because the body-fluid receiving hole 44b is formed instantaneously only by unfolding the folded interlabial pad 44. It should be noted that FIG. 15 shows an example of interlabial pad 44 composed of two absorbent sheet bodies 46a, 46b.

(9) The interlabial pad according to any one of (6) to (8), comprising a bellows portion where a plurality of said tucking portions are formed on at least one of the side wall and the bottom portion of said body-fluid receiving hole.

Not only single, the tucking portion may be formed in plurality respectively on the side wall and bottom portion, into bellows. For instance, the interlabial pad 44 shown in FIG. 16 has bellows portion 48 where a plurality of tucking portions 47 is formed only on the side wall portion of the body-fluid receiving hole 44b. As such, when bellows portion 48 is formed in the body-fluid receiving hole 44b, the surface area of the body-fluid receiving hole can be increased by far, compared to a case of forming a single tucking portion 47, preserving the convenience of carrying or handling, and the body fluid (especially menstrual blood) absorption capacity can further be improved.

The bellows portion can be formed on one or both of side wall and bottom portion of the body-fluid receiving hole. For instance, as shown in FIG. 17, it is also possible to form the bellows portion 48 on both of side wall and bottom portion.

(10) The interlabial pad according to any one of (1) to (9), wherein a maximum depth of said body-fluid receiving hole ranges from 10 mm to 60 mm.

As mentioned above, the body-fluid receiving hole is made as the one projecting on the garment side of the pad; however, it is preferable to compose so that it exists in a space from the labia to the underwear, and its maximum depth may be decided on consideration of the menstrual absorbing capacity. From the aforementioned point of view, the maximum depth is preferably in a range of 10 to 60 mm, more preferably in a range of 15 to 50 mm, and especially preferable in a range of 20 to 40 mm. By setting the maximum depth in this range, it becomes possible to reduce the contact between the labia and the pad by securing a clearance with the labia, improving the absorbing capacity in the body-fluid receiving hole. Moreover, the contact area between the pad and the garment (underwear) can be reduced, serving to mitigate the feeling of wrongness during the wearing.

(11) The interlabial pad according to any one of (1) to (10), further comprising an adhesion portion surrounding an outer circumference of the opening portion of said body-fluid receiving hole is formed on the body side surface of said interlabial pad.

For instance, as shown in FIG. 18, by forming an adhesion portion 49 on the body side surface of the interlabial pad 44, the interlabial pad 44 can be adhered further firmly between interlabial or to the pudenda. Such mode prevents a gap from generating between the interlabial pad 44 and the body by a sudden posture change of the wearer, or the interlabial pad 44 from slipping off, and can reduce the danger of drop of the interlabial pad 44. Consequently, it becomes possible to conduct menstrual blood more securely to the body-fluid receiving hole 44b, and the wearer can perform a free activity in peace, without being constricted in her behavior.

(12) The interlabial pad according to any one of (1) to (11), wherein the interlabial pad is used for incontinence of urine.

The aforementioned interlabial pad can be used as an absorbent pad for incontinence of urine. In short, as both the ostium vaginae discharging menstrual blood and the urethral meatus for discharging urine are interposed between interlabia, menstrual blood not only can be absorbed but also urine.

Thus, according to the present invention, an absorbent pad effective for incontinence, especially for a slight incontinence can be obtained, as it can absorb urine between interlabia, especially near the urethral meatus.

(13) The interlabial pad according to any one of (1) to (11), wherein the interlabial pad is used for absorbing the vaginal discharge.

The aforementioned interlabial pad can be used for absorbing the vaginal discharge. In short, as the interlabial pad according to the present invention is pinched between interlabia for use, it can absorb the excretion (vaginal discharge) other than menstrual blood if it is discharged from the ostium vaginae, so it can be also used for this application (vaginal discharge absorption).

(14) The interlabial pad according to any one of (1) to (13), wherein the interlabial pad is an interlabial pad for use with a sanitary napkin.

Some of users of sanitary napkin (referred to simply as “napkin”, hereinafter) use several napkins overlaid when menstrual blood is abundant; however, it has caused difficulties that a coarse feeling or the like occurs during wearing it, and the presence of the pad could easily be perceived (outstanding) from outside the garment. Besides, napkins were overlaid on a portion not requiring the overlaying other than the vicinity of the vagina, provoking a rash and becoming musty. However, by using a pad and napkins at the same time, the sanitary article turns up to be overlaid only in the vicinity of the vagina, allowing to avoid the aforementioned inconveniences. Further, there is also an effect that the wearer needs not to carry around napkins of an outstanding size, because only pad can be exchanged without changing napkins.

(15) A wrapping body, comprising: an interlabial pad according to any one of (1) to (14) and a wrapping container for individual wrapping the interlabial pad.

If interlabial pads are wrapped individually, it becomes possible to carry pads one by one (by an individual pack). Such mode eases the transport, all the way keeping the pad clean, compared to a case where a plurality of pads are contained in a single wrapping container, and the mode serves to a simple handling.

(16) The wrapping body according to (15), wherein the interlabial pad is enclosed in said wrapping container so as to be folded to the garment side direction substantially along the central line in the longitudinal direction thereof.

“So as to be folded to the garment side direction” means to include a case where it is bent protruding to the garment side as shown in FIG. 19, in addition to a case where it is folded completely protruding to the garment side. Thus, the wrapping body 62 enclosing the interlabial pad 44 in a wrapping container 61, allows the wearer to fit the pad more rapidly and simply, because the folded body-fluid receiving hole 44b unfolds itself naturally when the wrapping container 61 is unwrapped.

(17) 17. A wrapping body, comprising: an interlabial pad according to any one of (6) to (14) and a wrapping container for individual wrapping the interlabial pad, wherein the interlabial pad is enclosed in the wrapping container, wherein the tucking portion or the bellow portion on said side wall of the interlabial pad is tucked in to store, at least said bottom portion is affixed temporarily on the inner wall of said wrapping container, and said body-fluid receiving hole is formed as the tucking portion or the bellow portion across said side wall spreads in accordance with unwrapping of the wrapping container.

As the aforementioned wrapping body, for instance, as shown in FIG. 20, a wrapping body 62 made by enclosing in a wrapping container 61 an interlabial pad 44 wherein bellows portion 48 is formed on the side wall of the body-fluid receiving hole 44b can be cited. In this wrapping body 62, the interlabial pad 44 is stored with the bellows portion 48 folded, and at least the bottom portion of the body-fluid receiving hole 44b is affixed temporarily to the inner wall of the wrapping container 61 with an adhesive such as pressure sensitive hot melt adhesive.

The wrapping container 61, being composed of an upper face sheet 61a and a lower face sheet 61b, wraps the interlabial pad 44, in such a way to pinch it from up and down. For such wrapping body 62, the body-fluid receiving hole 44b will be formed by developing the bellows portion 48 on the side wall of the body-fluid receiving hole 44b, when the wearer unwraps the wrapping container 61 in such a way to peel of the lower face sheet 61b from the upper face sheet 61a in the arrow direction, as shown in FIG. 21. Consequently, even an interlabial pad 44 with a large surface area of the body-fluid receiving hole 67, can be enclosed compactly by folding. In addition the body-fluid receiving hole 44b can be formed easily and rapidly in a series of operations for unwrapping the wrapping container 61.

### BRIEF DESCRIPTION OF THE DRAWINGS

FIG. 1 is an illustration showing a structure of an interlabial pad according to the present invention, (a) is a top view of the pad, and (b) is a cross sectional view along the line A-A′ of (a);

FIG. 2 is a perspective view showing a structure of a conventional interlabial pad;

FIG. 3 is a perspective view showing a structure of a conventional sanitary napkin;

FIG. 4 is a perspective view showing a structure of the interlabial pad according to the present invention;

FIG. 5 is a cross sectional view along the line A-A′ of the interlabial pad shown in FIG. 4;

FIG. 6 is a perspective view showing a cross section of the interlabial pad according to the present invention;

FIG. 7 is a process drawing showing the manufacturing process of the interlabial pad shown in FIG. 6;

FIG. 8 is a perspective view showing a cross section of the interlabial pad according to the present invention;

FIG. 9 is an explanatory illustration showing the manufacturing process of the interlabial pad shown in FIG. 8;

FIG. 10 is an explanatory illustration showing the manufacturing process of the interlabial pad shown in FIG. 8;

FIG. 11 is an explanatory illustration showing the manufacturing process of the interlabial pad shown in FIG. 8;

FIG. 12 is a perspective view showing a cross section of the interlabial pad according to the present invention;

FIG. 13 is a perspective view showing a cross section of the interlabial pad according to the present invention;

FIG. 14 is a cross sectional view along the line A-A′ of the interlabial pad shown in FIG. 13;

FIG. 15 is a cross sectional view of the interlabial pad in the folded state according to the present invention;

FIG. 16 is a cross sectional view of the interlabial pad according to the present invention;

FIG. 17 is a cross sectional view of the interlabial pad according to the present invention;

FIG. 18 is a perspective view showing the cross section of the interlabial pad according to the present invention;

FIG. 19 is a perspective view showing a structure of a wrapping body according to the present invention;

FIG. 20 is a cross sectional view showing a structure of the wrapping body according to the present invention;

FIG. 21 is a cross sectional view showing a structure of the wrapping body according to the present invention;

FIG. 22 is a cross sectional view showing a structure of an absorbing sheet body;

FIG. 23 is an illustration showing the experimental state for peeling strength measurement of an adhesive; and

FIG. 24 is an illustration showing the experimental state for shear strength measurement of the adhesive.

### DETAILED DESCRIPTION OF THE PREFERRED EMBODIMENT

Now, examples of the best embodiments of the present invention shall be described in detail referring to drawings. It should be noted that, in the following description, “interlabial pad” may sometimes simply referred to as “pad”.

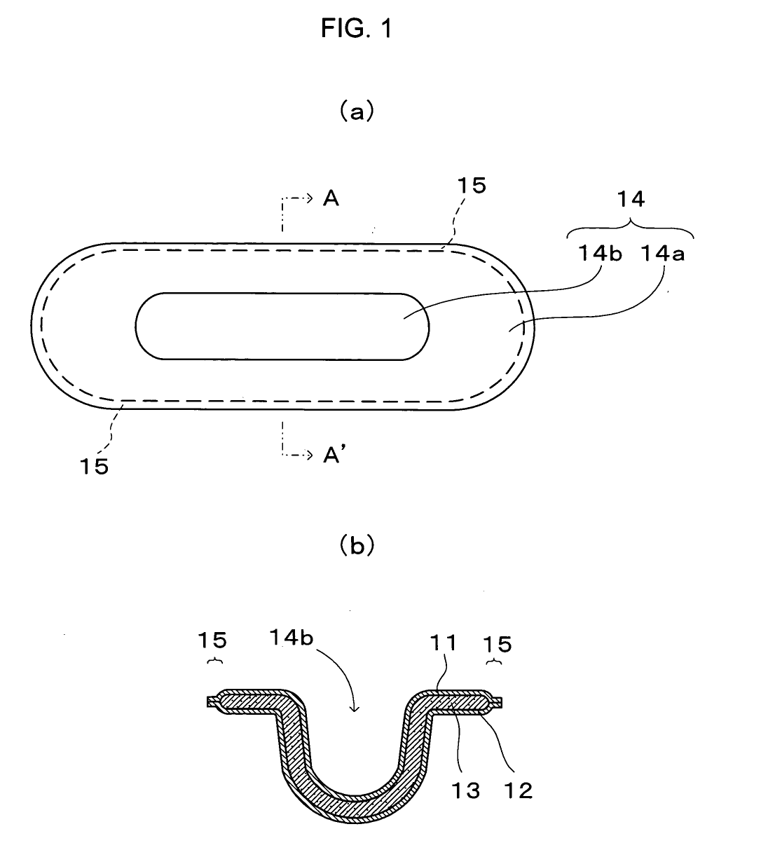

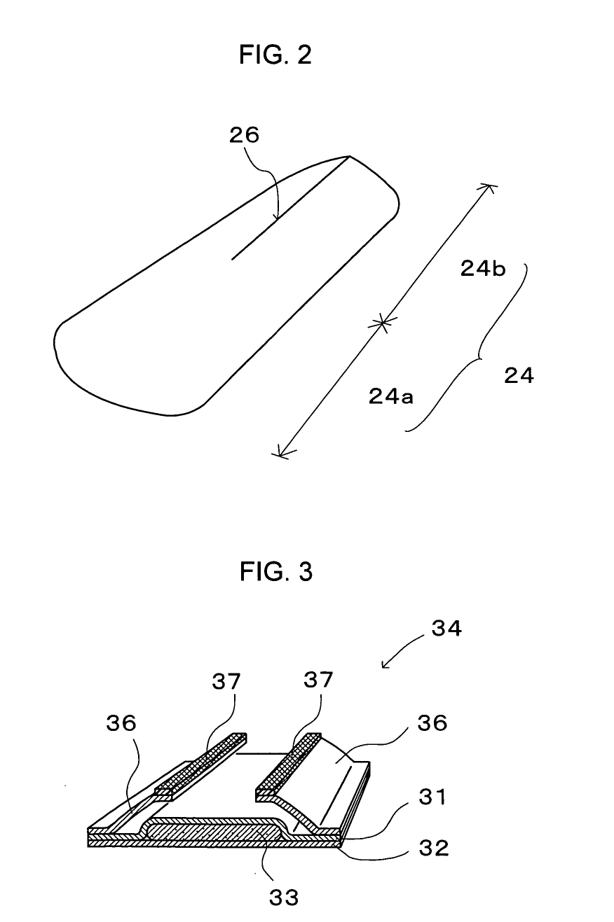

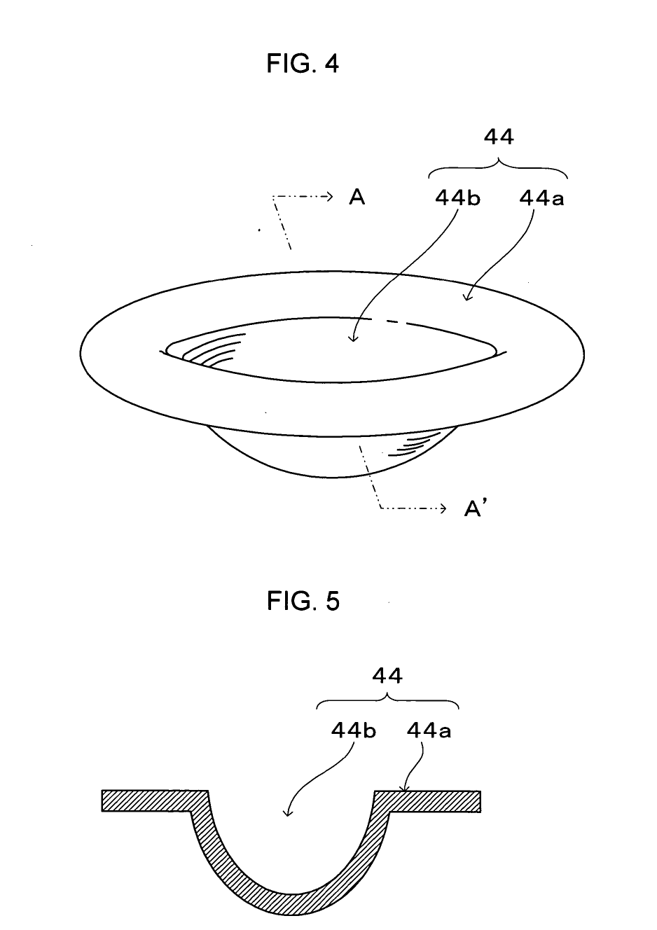

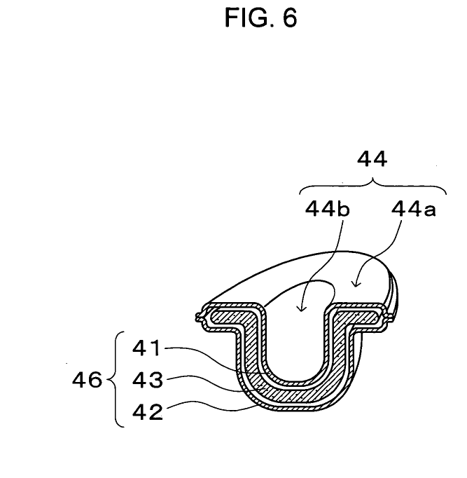

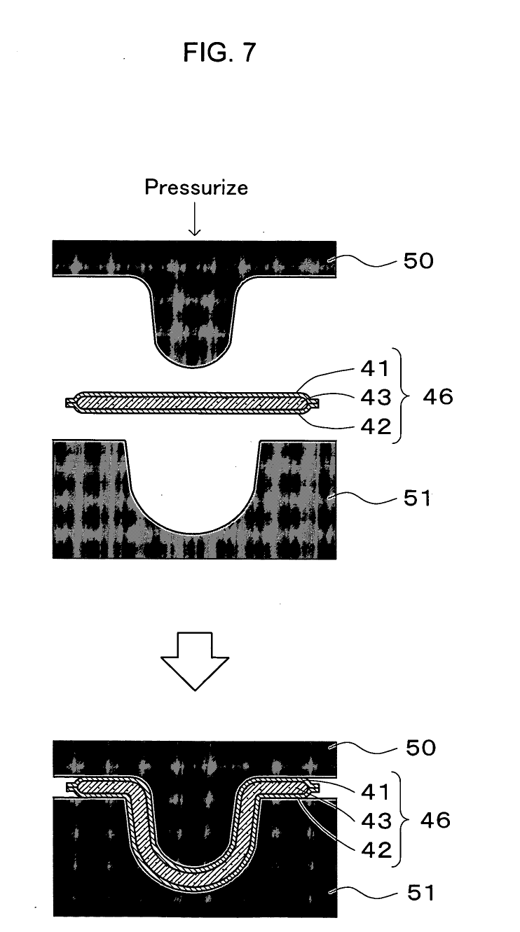

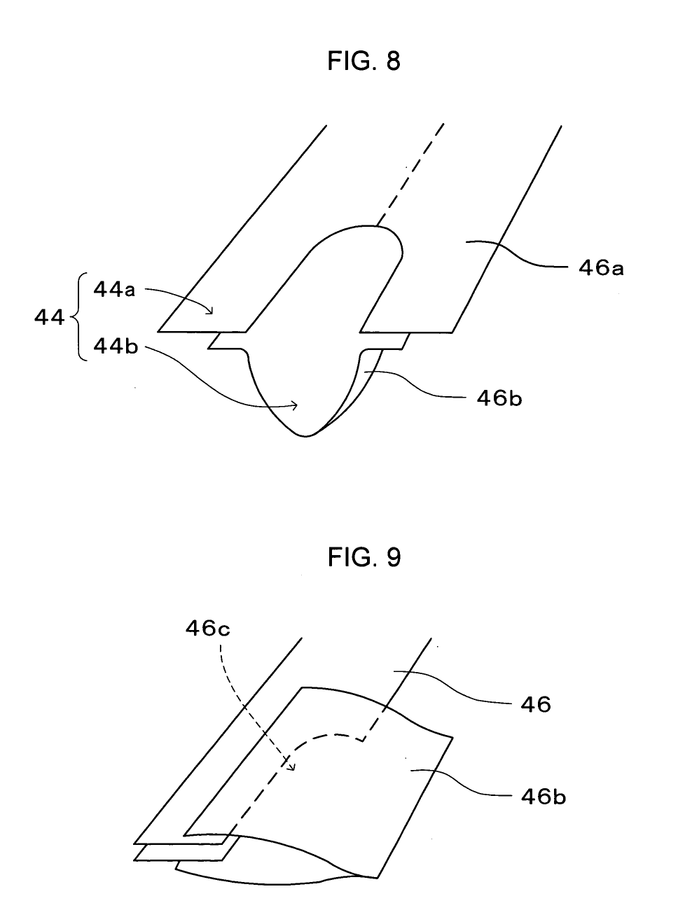

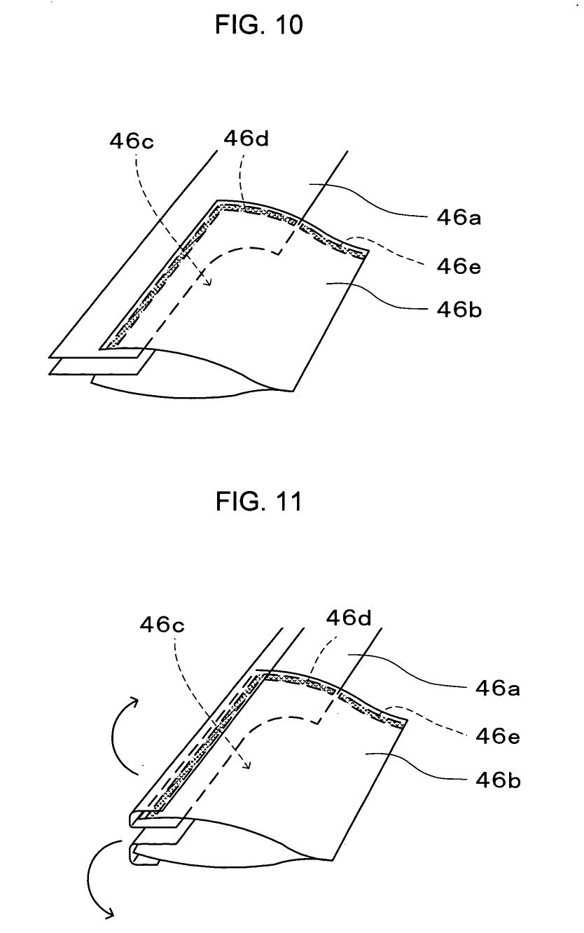

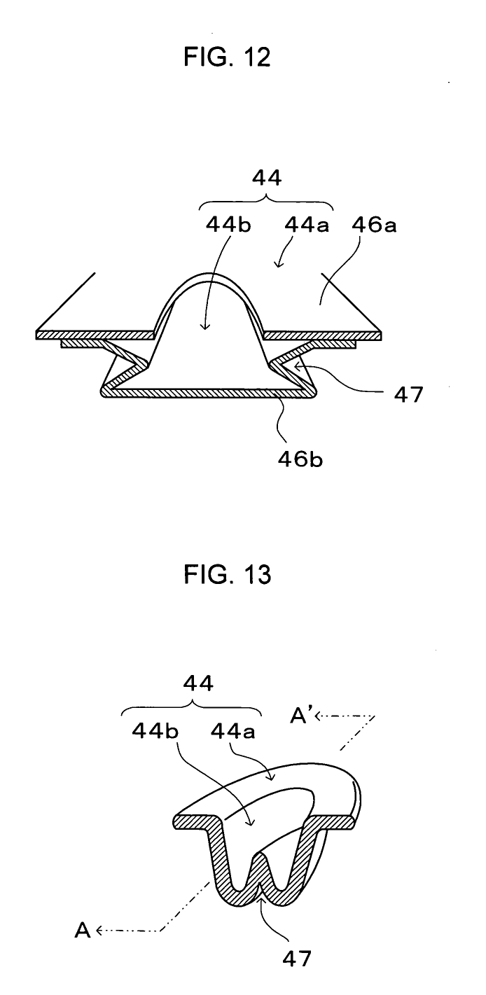

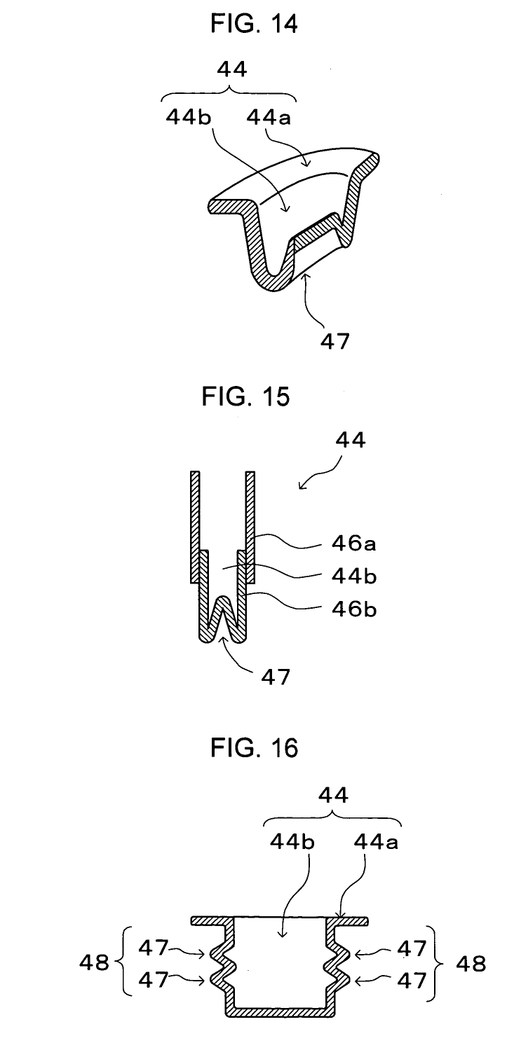

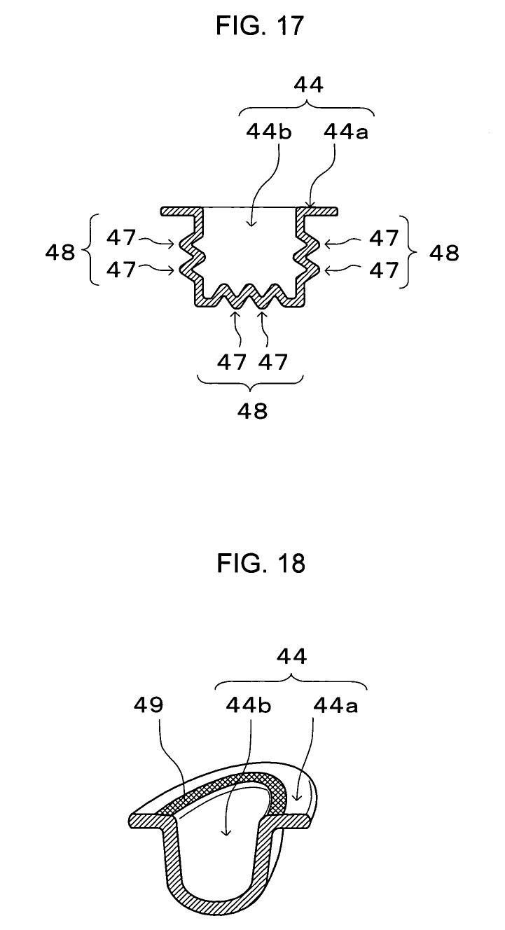

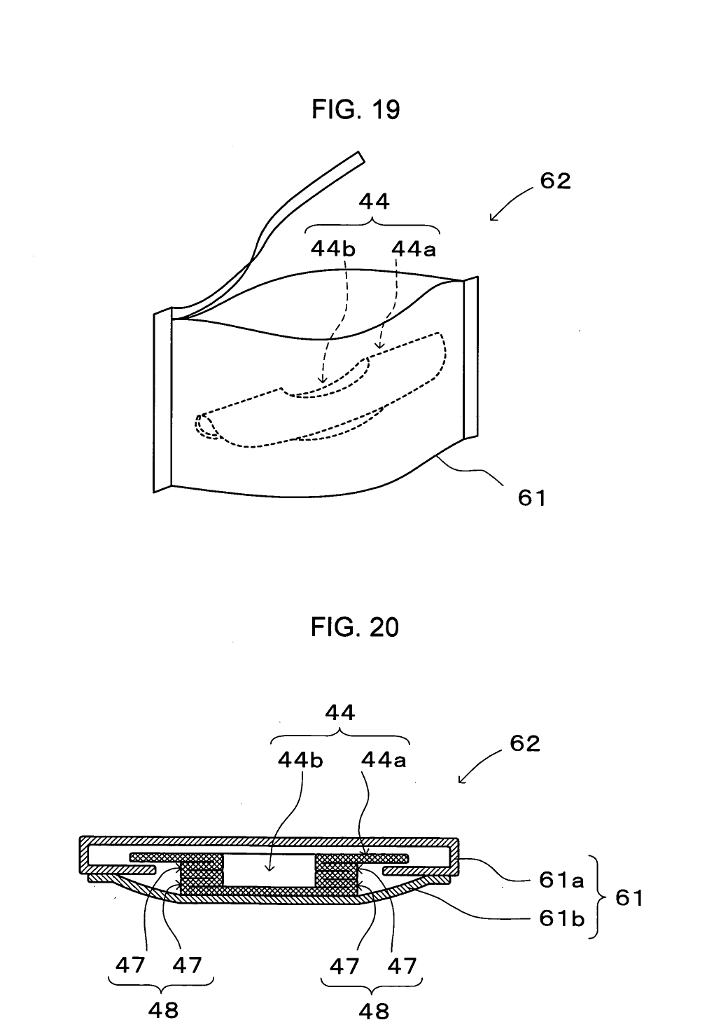

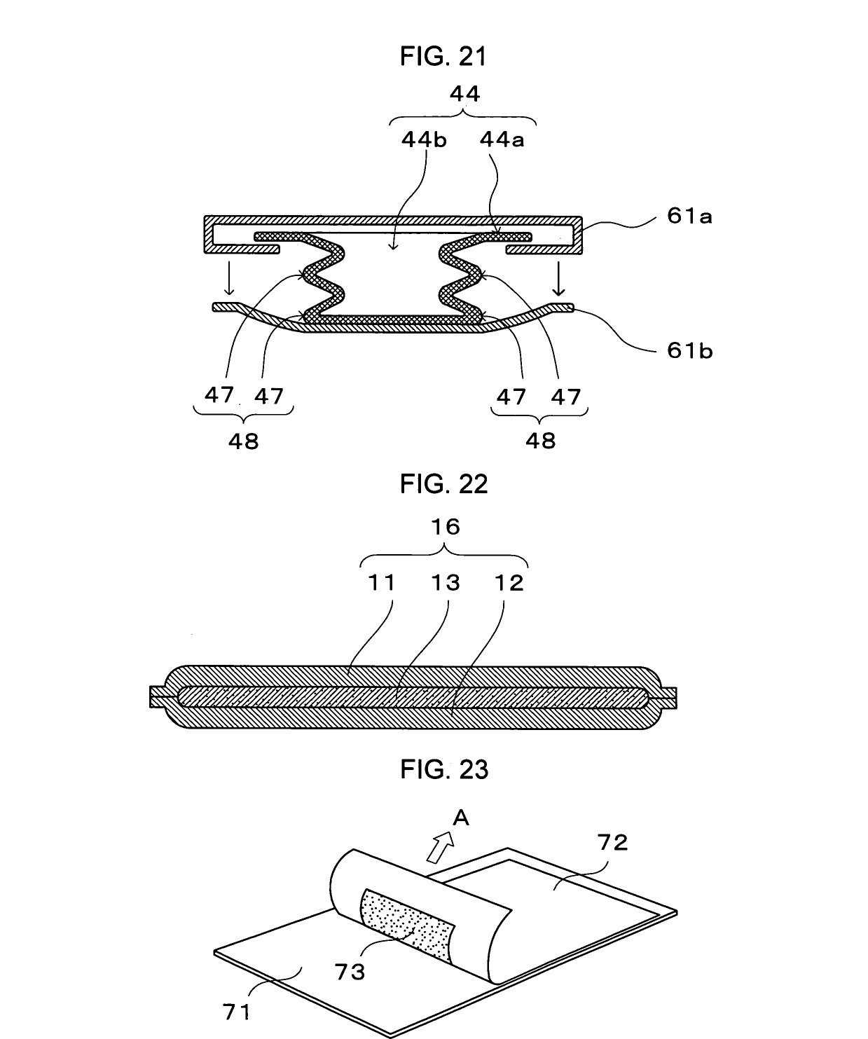

FIG. 1(a) is a top view showing the interlabial pad 14 according to the present invention, while FIG. 1(b) a cross sectional view along the line A-A′ of the interlabial pad 14 shown in FIG. 1(a).

[(A) Composition of a Basic Interlabial Pad]

A basic interlabial pad 14 of the present invention is provided with a body-fluid receiving hole 14b for storing menstrual blood, in the vicinity of the middle portion of a substantial flat area 14a. The body-fluid receiving hole 14b is a recess with a bottom portion corresponding to the garment side, and the size of the opening thereof is composed so as to envelop and fit to a pair of labia minora from outside.

The general shape of the interlabial pad 14 is not especially limited, provided that it is a shape appropriate for engaging with the labia, but it is preferable that the shape is substantially longitudinal and specifically, oval type, egg type, gourd type, drop type, or other shapes can be cited.

The interlabial pad 14 has preferably a length (dimension in the longitudinal direction of the pad) that would not interfere with the motion of the thigh or haunches of the wearer when engaged with the labia. Specifically, it is preferably in a range of 50 to 150 mm, and more preferably in a range of 60 to 130 mm, and especially preferably in a range of 80 to 120 mm. In addition, considering the stability in the case of wearing the pad, a width (lateral direction size of the pad) allowing hardly bring into contact with the thigh is preferable. To be specific, it is preferably in a range of 30 to 80 mm, and more preferably in a range of 40 to 60 mm.

The shape of the body-fluid receiving hole 14b may be selected conveniently from oval type, rectangular type, gourd type and so on; however, considering the morphologic compatibility with the labia, a longitudinal oval type as shown in FIG. 1(a) is preferable.

A pad is composed of a single or a plurality of absorbent sheet body 16 where a water permeable face side sheet 11 facing to the body side and a water permeable or impermeable back face side sheet 12 facing to the garment side are bonded in such a manner enclosing an absorbent body 13, as shown for instance in FIG. 22.

The face side sheet 11 and the back face side sheet 12 are bonded in such a manner enclosing the absorbent body 13, by heat emboss processing and/or adhesion by a hot melt type adhesive across a peripheral portion 15 as shown in FIG. 1.

In case of bonding the peripheral portion of the face side sheet 11 and the back face side sheet 12, it is preferable to bind so that the absorbent body 13 might not be pinched in the bonding portion. Because, if the absorbent body 13 is pinched in the bonding portion, it is feared that the peripheral portion 15 of the interlabial pad 14 turns up to be hard, generating a foreign feeing or discomfort during the wearing. For instance, the face side sheet 11 and the back face side sheet 12 may be bonded into a bag shape in a state where an opening portion is left partially beforehand and the absorbent body 13 may be packed into the bag-shaped portion, so that the absorbent body 13 would not be pinched in the bonding portion. It should be noted that the dimension of the absorbent body 13 may be equivalent to the interlabial pad 14, it may also be composed slightly smaller than the interlabial pad 14, in a way to permitting to secure an interval of 2 to 10 mm from the outer contour of the interlabial pad 14, so that the absorbent body 13 would not be pinched in the bonding portion.

Besides, the absorbent body 13 is in a state of being stuck to both sheets, in order to prevent it from separating from the face side sheet 11 and the back face side sheet 12. When the pad 14 is used (when wet with menstrual blood), the aforementioned members separate easily, and in order to prevent this, it is preferable to adhere the aforementioned respective members by heat emboss processing. The heat emboss processing can adhere in dot shape or screen pattern shape, and improve the use strength (when wet) without killing the water permeability, by making the area ratio of the emboss portion in a range of 3 to 20% of the total area.

[Surface Side Sheet]

Concerning the water permeable surface side sheet, a soft one less irritant to the sensitive labia or labial inner wall may be selected among sheet-shaped objects of liquid-permeable structure such as woven fabric, nonwoven fabric or perforated plastic sheet an so on. From such viewpoint, it is preferable to select through air nonwoven fabric, point bond nonwoven fabric and so on. In addition, spun lace nonwoven fabric formed by the water-flow interlacing treatment can be used advantageously in a point that it is excellent in drape property, and melt blown nonwoven fabric made into sheet by blowing out melt resin into extremely fine fiber by air pressure, in a point that it is composed of fine and soft fiber.

As fiber material constituting the aforementioned sheet-shaped object, any of natural fiber, regenerated fiber, semi-synthetic fiber or synthetic fiber can be used. As example of natural fiber, natural cellulose fiber of which fiber length is in a range of 5 to 25 mm can be cited, as example of regenerated fiber, regenerated cellulose of which fiber length is in a range of 38 to 51 mm, and of which finesse is in a range of 1.3 to 3.3 dtex, as example of semi-synthetic fiber, semi-synthetic cellulose, and as example of synthetic fiber, polyethylene (PE), polypropylene (PP), polyethylene terephthalate (PET) and so on can be cited. The synthetic fiber may be a bicomponent fiber having a sheath-core structure with core in PP or PET, and sheath in PE, or the like.

Among the aforementioned fiber materials, those composed of a fiber material having a structure that could conduct menstrual blood discharge from the ostinum vaginae to the absorbent body are preferable. For example, synthetic fibers having a cross section form such as Y type, C type, geometric type in fiber section, or natural cellulose, regenerated cellulose (viscose rayon), semi-synthetic cellulose (acetate or the like) and so on can be cited.

Concerning a more specific composition of the face side sheet, the spun lace nonwoven fabric wherein 40 to 90 weight % of regenerated cellulose and 10 to 60 weight % of natural cellulose are adjusted in a range of 25 to 50 g/m² (preferably 25 to 35 g/m²) by a specific weight per unit by the water-flow interlacing treatment can be cited. Also, one wherein a so-called “tow” (obtained by refining continuous fascicule) is adjusted in a range of 50 to 200 g/m² is preferable, in the point that it constitutes a soft sheet-shaped object excellent in flexibility in respect to the labia.

[Absorbent Body]

Any absorbent body that is hydrophilic and capable of absorbing and retaining liquid (menstrual blood) is acceptable, and it is preferably bulky, hard to deform and low in chemical stimulation. More preferably, it is high in flexibility considering that the pad is engaged with the pad.

As fiber material composing the absorbent body, selected from the aforementioned view point, for instance, wood pulp, natural cellulose fiber, regenerated cellulose fiber, and hydrophilic treated hydrophobic synthetic fiber can be cited, and they can be used solely or by mixing conveniently. Also, acrylic acid, graft copolymer of acrylic acid and starch, starch, carboxymethyl cellulose, particulate super absorbent polymer or super absorbent polymer fiber such as a foamed super absorbent polymer (foamed SAP) and the like, or a synthetic fiber (including a filament and a bicomponent fiber with a sheath-core structure) may be mixed.

In addition to the aforementioned, sheet-shaped matter of crushed matter of tissue, hydrophilic treated foam sheet, hydrophilic treated melt blown nonwoven fabric, fiber web comprising mainly hydrophilic fiber, spun lace nonwoven fabric obtained by forming a fiber web into a sheet, thermal bonded nonwoven fabric or other nonwoven fabric material, the aforementioned “tow”, expanded foam comprising mainly hydrophilic treated synthetic rubber or cellulose sponge comprising mainly cellulose can be used advantageously as the absorbent body.

Besides, considering the compatibility with the labia during the wearing, for instance, a laminate sheet of perforated plastic sheet where a liquid duct is formed and spun lace nonwoven fabric or crushed matter of the laminate sheet may also be used.

As for a more concrete composition of absorbent body, a sheet-shaped matter wherein a spun lace nonwoven fabric is formed by making a fiber web blending by a ratio of 60 to 90 weight % of regenerated cellulose fiber of 2.2 dtex in finesse and 10 to 40 weight % of natural cellulose into a sheet form through the water-flow interlacing treatment, and adjusted in a range of 50 to 500 g/m² (preferably 150 to 400 g/m²) in specific weight per unit by overlay it solely or in plurality.

[Back Face Side Sheet]

Concerning the material used for the back face side sheet, those similar to the face side sheet can be used in case of using water permeable material. In this case, it is preferable to use the pad with a sanitary napkin (pad used with sanitary napkin).

Besides, in case of using water impermeable material for the back face side sheet, it is possible to prevent menstrual blood held by the absorbent body from leaking out of the interlabial pad. The water impermeable material is preferably high in flexibility, in addition to the water impermeability. To be more specific, film or others using polyolefin resin (for example, low density polyethylene (LDPE), linear low density polyethylene (LLDPE), or high density polyethylene (HDPE)) as raw material or a film using synthetic rubber (for instance, styrene-ethylene/butadiene-styrene block copolymer (SEBS), styrene-isoprene-styrene block copolymer (SIS), styrene-butadiene-styrene block copolymer (SBS), urethane, and so on) as raw material can be cited.

The water impermeable material is preferably moisture-permeable. Because, this can reduce the humidity during the fitting, and it becomes possible to lower the discomfort during the wearing. For instance, so-called moisture-permeable film forming fine cells by blending and elongating inorganic fillers, imperforate moisture-permeable film made of polyester/polyether block copolymer resin, perforated moisture-permeable film having such aeration hole that a liquid leaks hardly and so on can be used advantageously.

[Adhesion Portion]

The adhesion portion may be arranged in planar, dot, network or linear shape. The position of the adhesion portion is not especially limited provided that it can be affixed to the body; however, considering the labial vicinity, particularly the presence of pubes on the forward portion of the labia, it is disposed preferably linearly in the vicinity of both side portions of the pad in a width of the order of 1 to 5 mm.

The “adhesion portion” can be formed by applying adhesive to the face side sheet. As for adhesive that can be used in the present invention, gel adhesive or the like comprising water-soluble polymer, cross-linking agent, plasticizer, and moisture can be cited. To be more specific, examples of water-soluble polymer comprise gelatin, sodium polyacrylate, polyvinyl alcohol, carboxylmethyl cellulose and so on, examples of the cross-linking agent comprise water soluble metallic salt such as calcium chloride and magnesium sulfate, and examples of plasticizer comprise glycerin, wax, paraffin, or others.

Other than them, pressure sensitive hot melt adhesives can also be used as adhesive for forming the adhesive portion. The pressure sensitive hot melt adhesive can be obtained by using synthetic rubber resin such as styrene-isoprene-styrene block copolymer (SIS), styrene-butadiene-styrene block copolymer (SBS), styrene-ethylene/butadiene-styrene block copolymer (SEBS), styrene-ethylene/propylene-styrene block copolymer (SEPS) as principal component, and melt blending of terpene resin, rosin resin or other tackifier and wax or other plasticizer therein.

Further, silicone resin type adhesives can also be used. As the silicone resin type adhesive, the mixture comprising mainly silicone resin and fluorocarbon resin, made by mixing cross-linking agent of platinum, molybdenum, antimony or other metal salt and plasticizer such as ester type wax, glycerin, machine oil or the like.

Thus, various kinds of adhesive for forming the adhesive portion exist; however, considering the application stability, it is preferable to use a pressure sensitive hot melt adhesive. As for the pressure sensitive hot melt adhesive presenting a good application stability, one of melt blending of 15 to 25 weight % of SEBS, 15 to 35 weight % of plasticizer and 40 to 70 weight % of tackifier can be cited. For this pressure sensitive hot melt adhesive, anti-oxidant or anti-fluorescent agents can be added in a range of 0.1 to 1.0 weight %.

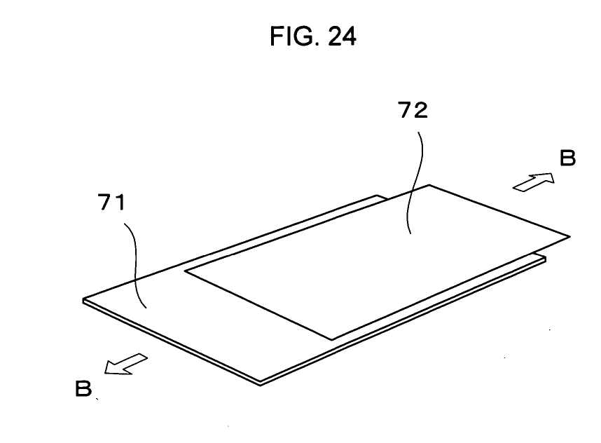

An example of valuation method of this adhesive force shall be described. Such valuation method consists in measuring the peel strength of the adhesive (FIG. 23) and the shear strength of the adhesive (FIG. 24), using a constant speed extension tensile test machine and a stainless steel board 71 of 80 mm in length ×50 mm in width. For performing the valuation test, beforehand, an adhesive 73 is applied so that the width becomes 25 mm and the length 50 mm on a polyethylene film 72 of substantially same size as the stainless steel board 71 and left under the room temperature (20° C.) for 30 minutes. Then, the polyethylene film 72 is stacked and put lightly so that the adhesive 73 comes into contact with the stainless steel board 71 and a roller is passed once (only one way) with an pressing force of 30 g/cm². Thereafter, it is left under the room temperature (20° C.) for 30 minutes for preparing the test piece.

The portion of polyethylene film 72 of the test piece prepared as mentioned above, is peeled off (180° peeling-off) by drawing in the direction of the arrow A shown in FIG. 23 in the peel strength test and it is drawn in the direction of the arrow B shown in FIG. 24 in the shear strength test. Nevertheless, as for the test condition, the speed of testing rate of stressing is set to 100 mm/min.

In case of measuring by the aforementioned method, the measured value of peel strength is preferably 100 to 2000 mN/25 mm, and the measured value of shear strength 3000 to 15000 mN/25 mm. The burden to the wearer's skin is taken into consideration.

It should be noted that the adhesive portion is preferably covered with a sheet made by coating a thin paper used generally as separate paper with silicone resin, or a sheet made by coating a plastic film with silicone resin. By so doing, the adhesion portion is prevented from soiling or peeling off during the storage.

[(B) Composition of Interlabial Pad Afforded with Biodegradability/Water Dispersibility/Water Solubility]

The interlabial pad of the present invention is preferably composed of biodegradable material and/or water dispersible and/or water soluble material. This is because the pad can be discarded simply and cleanly, and the waste in the toilettes can be reduced, as such pad can be dropped and flushed as it is after the usage.

In the present specification, the “biodegradability” means that a substance is discomposed into gas such as carbon dioxide or methane and so on, water and biomass under the anaerobic or aerobic condition, according to the process of the Nature, in the presence of actinomycetes including bacteria, and other microorganisms, and that the biodegradability (biodegradation speed, biodegradation degree, and others) of the substance corresponds to that of a naturally created material such as fallen leaves or a synthetic polymer generally recognized as diodegradable in the identical environment. The “water dispersibility” means the same thing as the water degradability and indicates the property that fibers each other are dispersed into small fragments that would not obstruct as least an ordinary toilettes piping, in a quantity of water or water flow, though not affected by a limited amount of moisture (menstrual blood) during the use. The “water solubility” indicates the property of solution in a large quantity of water or water flow, though not affected by a limited amount of moisture (menstrual blood) during the use.

[Face Side Sheet]

Concerning material of the water permeable face side sheet, both natural fiber and chemical fiber can be used. As examples of natural fiber, crushed pulp, cotton or other cellulose, airlaid pulp or others made by their chemical bonding with water-soluble resin can be cited. Examples of chemical fiber include, other than hydrophilic treated regenerated cellulose such as rayon, fibril rayon or the like, and chemical fiber such as PE, PP, PET, ethylene acetate vinyl copolymer or the like, so-called biodegradable fiber such as poly lactic acid, polybutylene succinate and so on. Besides, carboxymethyl cellulose, polyvinyl alcohol or others having the water solubility may also be used. Among the material, it is preferable to use pulp, cotton or other celluloses, rayon of other regenerated celluloses, so-called biodegradable fiber such as poly lactic acid.

It should be noted that the aforementioned materials can be used by forming into a web or nonwoven fabric, solely or in mixture. The web forming of so-called biodegradable fiber such as polylactic acid, polybutylene succinate and so on may be performed by the card method, spun bond method, melt blown method, airlaid method or either dry method or wet method, or by combining a plurality thereof. Concerning the bonding method, thermal bonding, needle punch, chemical bonding or other methods can be cited, but it is not particularly limited to these methods. Besides, a spun lace formed in a sheet shape by the water-flow interlacing treatment may also be used.

By way of example, the forming method for affording with the water dispersibility, method of forming fiber into a sheet shape by hydrogen bonding of fibers each other for obtaining hydrolyzate paper, method of binding fibers each other by a water-soluble binder and forming into a sheet-shape for obtaining hydrolyzate paper, method of forming fiber into a sheet shape by confounding fibers for obtaining hydrolyzate paper, and so on can be cited.

It should be noted that, in order to afford with a satisfactory water dispersibility, the fiber length is set preferably in a range of 2 to 51 mm and, more preferably, in a range of 2 to 10 mm. Moreover, for the coexistence of the water dispersibility and such a strength that would not generate a damage during the use, the finesses (size) is selected preferably from a range of 1.1 to 4.4 dtex. Below this range, though the water dispersibility will be satisfactory, fluffing and fluff drop occur often when dry, and on the other hand, in case of exceeding this range, the water dispersibility drops remarkably.

The specific weight per unit of the face side sheet is preferably set in a range of 20 to 60 g/m². In addition, the breaking strength of the face side sheet (breaking strength in case of constant rate of specimen extension with the length of specimen between grips of 100 mm and speed of testing rate of stressing of 100 mm/min) is necessarily at least 800 mN/25 mm both in the longitudinal and lateral direction and it is preferable to set in a range of 1000 to 7000 mN considering the flexibility during the fitting.

Concerning a more concrete composition of the face side sheet, for instance, a wet forming spun lace nonwoven fabric wherein rayon fiber of 1.1 to 4.4 dtex and 5 to 10 mm in length and wood pulp are blended with a weight ratio of 90:10 to 70:30 and the specific weight per unit is adjusted to 25 to 40 g/m², and the thickness to 0.2 to 0.5 mm, and so on can be cited. It should be noted that it is also possible to perforate a plurality of holes in the face side sheet in order to improve substantially the permeability (water permeability) of menstrual blood, or to afford an image of easy filtering of menstrual blood. In this case, they may be formed in a range of 0.5 to 1.5 mm in hole diameter, and in a range of 3 to 20% in hole portion area ratio (perforation ratio to the whole area).

[Absorbent Body]

As for the material of the absorbent body, those similar to the water permeable face side sheet can be used. Further, it is also possible to use sodium alginate, starch, caroboxymethyl cellulose or other absorbent material or particulate object or fiber-like object of high-absorbent polymer solely or in mixture with the material, through the forming thereof.

Concerning a concrete composition of the absorbent body, one wherein wood pulp is laminated by a specific weight per unit of 150 to 500 g/m² and sealed in a tissue, and adjusted to a thickness of 2 to 20 mm by a press device, and so on can be cited. It is also possible to increase the menstrual blood absorption or retention capacity, by blending starch or other absorbent material in the aforementioned absorbent body with a ratio of 5 to 30 g/m².

[Back Face Side Sheet]

As material of water impermeable back face side sheet, metylcellulose, hydroxyethylecellulose, caroboxymethyl cellulose or other derivates of cellulose, polyvinyl alcohol, sodium alginate, sodium poly acrylate, polyacrylic ether, polyvinyl pyrolidone, copolymer of isobutylene and anhydrous maleic acid or other water-soluble polymers, or, poly lactic acid, polybutylene succinate, starch, dextrin or other biodegradable polymers can be cited.

Concerning the materials, they can be formed into a melt blown nonwoven fabric or film sheet solely or blending with the material. Further, silicone resin or other water repellent material can be applied to or blended with the film sheet, or, nonwoven fabric made of the material and laminated may also do.

As for a concrete composition of the water impermeable back face side sheet, for instance, one wherein silicone or fluorine compound is applied to a thickness of 0.5 to 5 μm to at least one face, and more preferably to both faces, of a film made by adjusting polyvinyl alcohol by a specific weight per unit of 20 to 50 g/m² or others can be cited.

[Mini Sheet Piece]

As for mini sheet piece, film made of polyvinyl alcohol, or, laminated material of film made of polyvinyl alcohol and tissue, and so on can be cited.

[Bonding Method]

Also, as for bonding method applied to the present invention, adhesion by polyvinyl alcohol or others having water solubility or water bloating tendency, heat seal, or bonding by hydrogen bond, or others boding method can be used solely or in combination conveniently.

INDUSTRIAL APPLICABILITY

As described hereinabove, in the interlabial pad according to the present invention, it is possible to prevent the side leakage and preserve the cleanliness function inherent in the interlabial pad and, furthermore, improve its absorption conservation capacity by far, because a body-fluid receiving hole is provided in the vicinity of the middle portion of the pad. In addition, the wrapping body according to the present invention encloses a tucking portion or bellows portion of the interlabial pad in a folded state, and at least a bottom portion of the body-fluid receiving hole is affixed temporarily to the inner wall of the wrapping container, therefore, the body-fluid receiving hole is formed as the tucking portion or bellows portion is unfolded along with the unwrapping of the wrapping container. Consequently, it becomes possible to store the interlabial pad compactly, and moreover, to take it out so that its function can be deployed instantaneously.

1. An interlabial pad, used by engaging with labia, comprising:
a water permeable face side sheet facing to a body side;
a water permeable or impermeable back face side sheet facing to a garment side; and
an absorbent body for absorbing body fluid,
wherein the face side sheet and the back face side sheet are bonded together to enclose the absorbent body, and
wherein a body-fluid receiving hole is provided in the vicinity of a middle portion of the interlabial pad, the body-fluid receiving hole being a recess having a bottom portion on the garment side, and having an opening with such a size that can fit a pair of labia minora so as to envelop them from outside.

2. The interlabial pad according to claim 1, wherein said body-fluid receiving hole projects toward the garment side of said interlabial pad.

3. The interlabial pad according to claim 1, wherein said body-fluid receiving hole is shaped so that the middle portion of said interlabial pad caves in.

4. The interlabial pad according to claim 1, comprising:
a sheet-shaped first absorbent sheet body having a through-hole of such a size that can fit a pair of labia minora so as to envelop them from outside; and a bag-shaped second absorbent sheet body serving as a portion of said body-fluid receiving hole,
wherein a garment side surface of said first absorbent sheet body and an inner side surface of the bag of said second absorbent sheet body are in contact each other and stuck together.

5. A method of producing an interlabial pad of claim 4, comprising the steps of:
folding the sheet-shaped first absorbent sheet body having, in the vicinity of the middle portion, the through-hole of such a size that can fit to a pair of labia minora so as to envelop them from outside substantially along a central line in the longitudinal direction;
pinching said first absorbent sheet body by the sheet-shaped second absorbent sheet body folded substantially along the central line in the longitudinal direction in a way to seal the through-hole portion of said folded first absorbent sheet body; and
forming a bag portion serving as the body-fluid receiving hole by sticking an abutment face of an outer peripheral side of the through-hole of in a surface of said first absorbent sheet body to an abutment face in a surface of said second absorbent sheet body.

6. The interlabial pad according to claim 1, wherein said body-fluid receiving hole has a tucking portion where said absorbent sheet body is tucked in.

7. The interlabial pad according to claim 6, wherein said tucking portion is formed on a side wall of said body-fluid receiving hole.

8. The interlabial pad according to claim 6 wherein said tucking portion is formed on a bottom portion of said body-fluid receiving hole.

9. The interlabial pad according to claim 7, comprising a bellows portion where a plurality of said tucking portions are formed on at least one of the side wall and the bottom portion of said body-fluid receiving hole.

10. The interlabial pad according to claim 1, wherein a maximum depth of said body-fluid receiving hole ranges from 10 mm to 60 mm.

11. The interlabial pad according to claim 1, further comprising an adhesion portion surrounding an outer circumference of the opening portion of said body-fluid receiving hole is formed on the body side surface of said interlabial pad.

12. The interlabial pad according to claim 1, wherein the interlabial pad is used for incontinence of urine.

13. The interlabial pad according to claim 1, wherein the interlabial pad is used for absorbing the vaginal discharge.

14. The interlabial pad according to claim 1, wherein the interlabial pad is an interlabial pad for use with a sanitary napkin.

15. A wrapping body, comprising:
an interlabial pad according to claim 1; and
a wrapping container for individual wrapping the interlabial pad.

16. The wrapping body according to claim 15, wherein said interlabial pad is enclosed in said wrapping container so as to be folded to the garment side direction substantially along the central line in the longitudinal direction thereof.

17. A wrapping body, comprising:
an interlabial pad according to claims 6; and
a wrapping container for individual wrapping the interlabial pad, wherein the interlabial pad is enclosed in the wrapping container,
wherein the tucking portion or the bellow portion on said side wall of the interlabial pad is tucked in to store, at least said bottom portion is affixed temporarily on the inner wall of said wrapping container, and said body-fluid receiving hole is formed as the tucking portion or the bellow portion across said side wall spreads in accordance with unwrapping of the wrapping container.
